(19) United States
(12) Patent Application Publication
(10) Pub. No.: US 2026/0281169 A1
(43) Pub. Date: Sep. 17, 2026

(54) HIGH-BANDWIDTH DATA STREAMING FOR DISTRIBUTED COMPUTING

(71) Applicant: Idea Sistemas Eletronicôs LTDA S.A., Campinas (BR)

(72) Inventors: Guilherme PAULINO, Campinas (BR); Álvaro Augusto COSTA, Campinas (BR); Gabriel ALVES DE AMORIM, Campinas (BR)

(21) Appl. No.: 19/218,896

(22) Filed: May 27, 2025

Related U.S. Application Data
(60) Provisional application No. 63/771,979, filed on Mar. 14, 2025.

(51) Int. Cl. H04L 65/60; A61B 6/42; H04N 19/176

(52) U.S. Cl. CPC H04L 65/60; A61B 6/4241; H04N 19/176

(57) ABSTRACT

A detector control unit (DCU) server includes an input memory buffer for storing raw image frames; an input communication interface for receiving the raw image frames from a photon-counting detector and providing the raw image frames to assigned memory locations of the input memory buffer; a bit-conversion processor for receiving the raw image frames from the input memory buffer, and including a plurality of processing threads for performing parallel bit-conversions, and wherein the bit-conversion processor is configured to convert pixel values of each raw image frame of the raw image frames from a raw bit depth to a higher bit depth to generate bit-converted image frames; and an output memory buffer for receiving the bit-converted image frames; and an output communication interface for receiving the bit-converted image frames from the output memory buffer, and streaming the bit-converted image frames out from the DCU server to an external network.

### CROSS-REFERENCE TO RELATED APPLICATION

This patent application claims priority to U.S. Provisional Patent Application No. 63/771,979, filed on Mar. 14, 2025, and entitled “HIGH-BANDWIDTH DATA STREAMING FOR DISTRIBUTED COMPUTING.” The disclosure of the prior Application is considered part of and is incorporated by reference into this patent application.

### TECHNICAL FIELD

The present disclosure relates generally to high-bandwidth data streaming for distributed computing and to streaming of photon-counting detector images.

### BACKGROUND

Modern, large-area detectors generate vast amounts of high-resolution image data at high-speed. A photon-counting detector is a type of sensor that detects and counts individual photons. As detector resolution and frame rates increase, so does challenges of efficiently handling image data, processing the image data, and/or storing the image data in real-time. Simply relying on more powerful hardware is not typically enough to efficiently handle, process, and/or store the image data in real-time. Software, integrated in hardware, should be designed to take full advantage of modern hardware features. Without the software, the hardware may experience bottlenecks that limit overall performance and render the hardware ineffective in handling, processing, and/or storing the image data in real-time.

### SUMMARY

In some implementations, a detector control unit (DCU) server includes an input memory buffer comprising a first plurality of memory locations, the input memory buffer configured to store raw image frames; an input communication interface configured to be communicatively coupled to a photon-counting detector for receiving the raw image frames from the photon-counting detector and provide the raw image frames to assigned memory locations of the first plurality of memory locations of the input memory buffer; a bit-conversion processor coupled to the input memory buffer for receiving the raw image frames from the input memory buffer, wherein the bit-conversion processor includes a plurality of processing threads for performing parallel bit-conversions, and wherein the bit-conversion processor is configured to convert pixel values of each raw image frame of the raw image frames from a raw bit depth to a higher bit depth to generate bit-converted image frames; an output memory buffer configured to receive the bit-converted image frames from the bit-conversion processor, the output memory buffer comprising a second plurality of memory locations for storing the bit-converted image frames; and an output communication interface configured to receive the bit-converted image frames from the output memory buffer, and stream the bit-converted image frames out from the DCU server to an external network.

In some implementations, a DCU server includes an input memory buffer comprising a first plurality of memory locations, the input memory buffer configured to store raw image frames; an input communication interface configured to be communicatively coupled to a photon-counting detector for receiving the raw image frames from the photon-counting detector and provide the raw image frames to assigned memory locations of the first plurality of memory locations of the input memory buffer; a bit-conversion processor coupled to the input memory buffer for receiving the raw image frames from the input memory buffer, wherein the one or more processors include a plurality of processing threads for performing parallel bit-conversions, and wherein the one or more processors are configured to convert pixel values of each raw image frame of the raw image frames from a raw bit depth to a higher bit depth to generate bit-converted image frames; an output memory buffer configured to receive the bit-converted image frames from the bit-conversion processor, the output memory buffer comprising a second plurality of memory locations for storing the bit-converted image frames; a compression engine configured to receive the receive the bit-converted image frames from the output memory buffer, and compress the bit-converted image frames into compressed image frames; a compression output memory buffer configured to receive the compressed image frames from the compression engine, the compressed output memory buffer comprising a third plurality of memory locations for storing the compressed image frames; and an output communication interface configured to receive the compressed image frames from the compression output memory buffer, and stream the compressed image frames out from the DCU server to an external network.

In some implementations, a method of processing raw image frames from a photon-counting detector includes receiving, by an input communication interface, the raw image frames from the photon-counting detector; providing, by the input communication interface, the raw image frames to assigned memory locations of a first plurality of memory locations of an input memory buffer; reading out, by a bit-conversion processor, the raw image frames from the input memory buffer to the bit-conversion processor; converting, by the bit-conversion processor, the raw image frames into bit-converted image frames, wherein converting the raw image frames into bit-converted image frames includes: processing each raw image frame of the raw image frames in a plurality of processing threads, of the bit-conversion processor, for performing parallel bit-conversions such that pixel values of each raw image frame of the raw image frames are converted from a raw bit depth to a higher bit depth to generate a bit-converted image frame; and streaming, by an output communication interface, the bit-converted image frames or compressed-versions of the bit-converted image frames to an external network.

### BRIEF DESCRIPTION OF THE DRAWINGS

FIG. 1 shows a system according to one or more implementations.

FIG. 2 shows diagrams of an operation flow of a circular buffer with two slots.

FIG. 3 shows an example of a process flow of the input communication interface.

FIG. 4 shows an example of an algorithm for converting 12-bit pixels to 16-bit using advanced vector extensions.

FIG. 5 shows an example of dividing image data into smaller chunks (data chunks) for parallel compression processing.

FIG. 6 shows an example of a bit shuffle.

FIG. 7 shows a sample synthetic image used for a compression benchmark.

FIG. 8 shows an example of a comparison of a compression speed with varying number of threads and sensor area, with and without applying bit-shuffling technique prior to compressing.

FIG. 9 shows an example of a threshold scan (left) and corresponding compression ratios (right) for a detector frame resolution, applying bit-shuffling before compression.

FIGS. 10A and 10B show an example of a streaming benchmark for photon-counting detector frames.

### DETAILED DESCRIPTION

The following detailed description of example implementations refers to the accompanying drawings. The same reference numbers in different drawings may identify the same or similar elements.

Some implementations provide a detector control unit (DCU) server for processing and streaming images received from a high-speed area detector. The DCU server may be a hardware system configured by a software system that, when executed, causes the hardware system to execute functions described herein. In some implementations, the high-speed area detector is an X-ray photon-counting detector.

The DCU server may utilize the software system to receive, process, and stream image data from high-speed area detectors. The software system may be optimized to take advantage of the hardware system, employing techniques such as multithreading, memory alignment, and vectorized algorithms. The software system may be deployed in the DCU server that is configured to be communicatively coupled to one or more X-ray photon-counting detectors. The software system may be stored in one or more hardware components of the DCU server and/or accessible to one or more hardware components of the DCU server. For example, the software system (e.g., software, such as an executable program code) may be stored in a memory of the DCU server. The DCU server, executing the software system, may be configured for detector control and image acquisition. Thus, the DCU server may control the one or more X-ray photon-counting detectors and may receive image data from the X-ray photon-counting detectors for processing and/or storing. By leveraging high-bandwidth networking and parallel processing techniques, the software system may enable real-time data streaming, making the DCU server suitable for applications that require immediate processing, such as scientific imaging and industrial inspection. The software system may improve image data throughput and may ensure scalability, allowing the DCU server to accommodate increasingly large detectors without significant performance degradation.

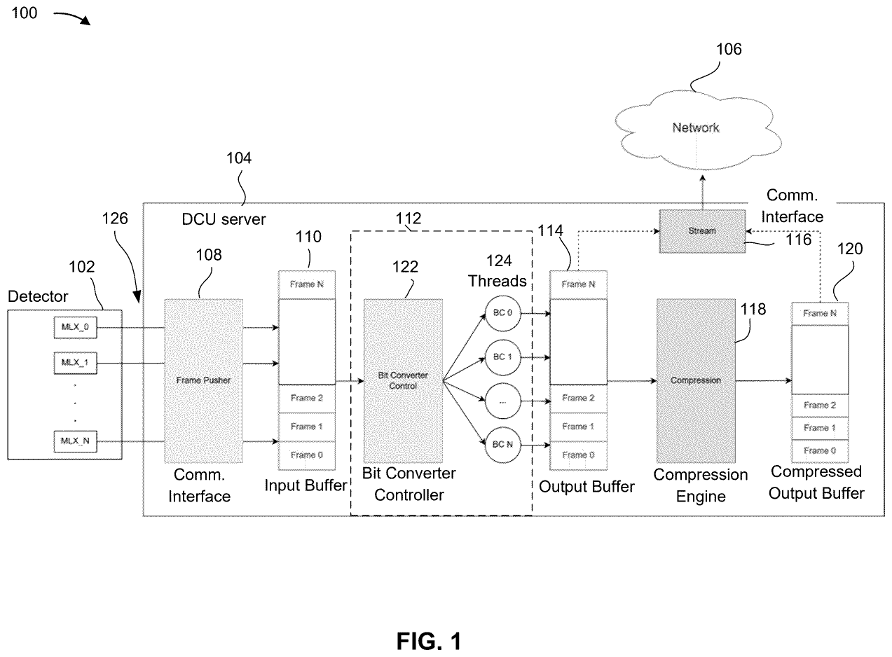

FIG. 1 shows a system 100 according to one or more implementations. The system 100 includes a detector 102, a DCU server 104, and an external network 106. The detector 102 may be a photon-counting detector, such as an X-ray photon-counting detector, that generates raw image frames based on photon-counting detection and transmits the raw image frame to the DCU server 104.

The DCU server 104 may include various hardware components, including an input communication interface 108 (e.g., a frame pusher), an input memory buffer, 110, a bit-conversion processor 112, an output memory buffer 114, an output communication interface 116, a compression engine 118, and a compression output memory buffer 120. The bit-conversion processor 112 may be a central processing unit (CPU). In some implementations, the bit-conversion processor 112 may include a bit converter controller 122 and a plurality of processing threads 124 (e.g., bit converter worker threads BC 0, BC 1, . . . , BC N) for performing parallel bit-conversions. The output communication interface 116 may stream bit-converted image frames or compressed data frames to the external network 106.

The DCU server 104 may store and execute software (e.g., a software system) that enables each hardware component to perform one or more designated functions. The hardware components may be arranged according to a software pipeline of the software. In some implementations, one or more of the hardware components are implemented as one or more processors configured to execute the software pipeline or portions of the software pipeline (e.g., portions of the software). In some implementations, the software pipeline may be executed by a central processing unit (CPU). Memory buffers may be implemented by (e.g., managed by) software, which may use random-access memory (RAM) to store temporary data.

The software system may be configured to receive images (e.g., image data) from the detector, process the images into a standard format, and stream the processed images (e.g., processed image data) over the external network 106, optionally applying compression. The detector 102 and the DCU server 104 may be connected by high-speed optical links. The input communication interface 108 may manage synchronization between the detector 102 and the input memory buffer 110. Thus, the input communication interface 108 may receive raw image frames (e.g., image data frames) from the detector 102 via one or more of the high-speed optical links. For example, the input memory buffer 110 may include a first plurality of memory locations (e.g., slots), and the input memory buffer 110 may store raw image frames received from the detector 102 and pushed out by the input communication interface 108 to the input memory buffer 110. Thus, the input communication interface 108 may be communicatively coupled to the detector 102 for receiving the raw image frames from the detector 102, and may provide the raw image frames to assigned memory locations of the first plurality of memory locations of the input memory buffer 110. Thus, the input communication interface 108 may manage synchronization between the detector 102 and the input memory buffer 110. In some implementations, the input communication interface 108 may to write the raw image frames into the input memory buffer 110 using a remote direct memory access (RDMA) protocol.

The input communication interface 108 may include a plurality of parallel optical links 126 for receiving the raw image frames. The input communication interface 108 may continuously poll the plurality of parallel optical links 126 for detecting a newly-received raw image frame. Additionally, the input communication interface 108 may check for a lost raw image frame by comparing sequence numbers or timestamps of the raw image frames. Thus, each raw image frame may be stored in a respective memory location of the input memory buffer 110.

The input memory buffer 110 may be a circular buffer. Additionally, the input memory buffer 110 may handle the raw image frames based on a first in, first out scheduling algorithm. In some implementations, the input memory buffer 110 may be a circular buffer that includes a first writer head index, a second writer head index, and one or more reader head indices. The first writer head index may indicate a first range of raw image frames currently being processed. The second writer head index follows the first writer head index and may indicate a second range of raw image frames that have been processed and are ready to be read out from the input memory buffer. Moreover, each reader head index of the one or more reader head indices may advance through the input memory buffer 110 to read out a raw image frame in the second range of raw image frames.

The bit-conversion processor 112 is coupled to the input memory buffer 110 for receiving the raw image frames from the input memory buffer 110. The bit-conversion processor 112 is configured to utilize the plurality of processing threads 124 for performing parallel bit-conversions such that the bit-conversion processor 112 converts pixel values of each raw image frame of the raw image frames from a raw bit depth to a higher bit depth to generate bit-converted image frames.

The bit converter controller 122 may firstly receive the raw image frames and forward the raw image frames to the plurality of processing threads 124 to balance workloads of the plurality of processing threads 124 and enable faster processing/bit conversion. Thus, the plurality of processing threads 124 are arranged in parallel threads. The bit-conversion processor 112 may serially receive the raw image frames from the input memory buffer 110.

The plurality of processing threads 124 may each include one or more bit converters that convert pixel values to the higher bit depth. The bit conversion of the plurality of processing threads 124 runs in parallel, with a workload being distributed by the bit converter controller 122 across the plurality of processing threads 124. The plurality of processing threads 124 may convert pixel values, from the raw bit depth to the higher bit depth, of a raw image frame of the raw image frames in parallel. The bit-conversion processor 112 may utilize single instruction, multiple data (SIMD) processing, associated with the plurality of processing threads, for converting the raw image frames into the bit-converted image frames. Additionally, the bit-conversion processor 112 may combine vectorized bitwise operations with shuffling and blending to covert the pixel values from the raw bit depth to the higher bit depth.

The bit-converted image frames are stored in the output memory buffer 114. For example, the output memory buffer 114 may include a second plurality of memory locations (e.g., slots) for storing the bit-converted image frames. Thus, each bit-converted image frames may be stored in a respective memory location of the output memory buffer 114. The output memory buffer 114 may also be a circular buffer. In some implementations, the output memory buffer 114 may include a first writer head index, a second writer head index, and one or more reader head indices. For example, the first writer head index may indicate a first range of bit-converted image frames currently being processed. The second writer head index follows the first writer head index and may indicate a second range of bit-converted image frames that have been processed and are ready to be read out from the output memory buffer. Additionally, each reader head index of the one or more reader head indices may advance through the output memory buffer 114 to read out a bit-converted image frame in the second range of bit-converted image frames.

In some implementations, the output communication interface 116 may receive the bit-converted image frames from the output memory buffer 114, and stream the bit-converted image frames out from the DCU server 104 to the external network 106.

The compression engine 118 may receive the bit-converted image frames from the output memory buffer 114, and compress the bit-converted image frames into compressed image frames. The compression engine 118 may be part of the CPU or may be a separate processor that may be designed for performing image frame compression. In some implementations, the compression engine 118 may utilize a lossless data compression algorithm in combination with bit shuffling to compress the bit-converted image frames into the compressed image frames. The lossless data compression algorithm may be a Lempel-Ziv-4 (LZ4) data compression algorithm however, using other lossless compression algorithms are possible. In some implementations, the compression engine may, for each bit-converted image frame of the bit-converted image frames, divide a bit-converted image frame into chunks for parallel processing of the chunks; compress the chunks, in separate compression threads of a plurality of compression threads, into compressed blocks; write each compressed block of the compressed blocks into an intermediate compression buffer; and concatenate the compressed blocks into a compressed image frame.

Accordingly, the compression engine 118 may receive the bit-converted image frames from the output memory buffer 114 in order to reduce a data volume and optimize network bandwidth usage and/or local storage performance of the DCU server 104. The compression engine 118 may compress the bit-converted image frames into compressed image frames. The compression engine 118 may use a lossless compression algorithm, that prioritizes fast compression and decompression, to compress the bit-converted image frames.

The compression output memory buffer 120 may receive the compressed image frames from the compression engine 118. The compressed output memory buffer 120 may include a third plurality of memory locations (e.g., slots) for storing the compressed image frames (which also have been bit-converted). The compression output memory buffer 120 may be a circular buffer that has a first writer head index, a second writer head index, and one or more reader head indices.

The output communication interface 116 may receive the compressed image frames from the compression output memory buffer 120, and stream the compressed image frames out from the DCU server 104 to the external network 106. Thus, the output communication interface 116 may be used to selectively stream the bit-converted image frames or the compressed image frames out from the DCU server 104 to the external network 106. In some cases, the output communication interface 116 may selectively stream the bit-converted image frames or the compressed image frames out from the DCU server 104 to the external network 106 based on an asynchronous messaging mechanism.

The output communication interface 116 may transmit standard bit depth images (e.g., the bit-converted image frames) or compressed images (e.g., compressed image frames) from the output memory buffer 114 or the compressed output memory buffer 120, respectively, over the external network 106. The output communication interface 116 may provide a flexible application programming interface (API) for distributed system integration, facilitating interoperability with control systems commonly used to operate high-speed detectors, such as EPICS, TANGO, or any other open or proprietary distributed control system.

The output communication interface 116 may stream images over the external network 106. The output communication interface 116 may use an asynchronous messaging library, such as ZeroMQ, to ensure seamless integration with third-party applications, regardless of a software platform used to execute the third-party applications. As long as a client device has network access, the client device may receive the images from the output communication interface 116 (e.g., from the external network 106), enabling flexible and efficient data distribution. The output communication interface 116 may transmit image in a standard format, from the output memory buffer 114, or in a compressed format, from the compressed output memory buffer 120.

A data structure used throughout the software pipeline is the circular buffer, also known as a ring buffer. This data structure is a fixed-size buffer that operates as if its endpoints are connected, allowing for continuous data flow without the need for explicit memory shifting. Circular buffers are widely used in high-performance applications due to their efficiency in managing streaming data, reducing memory fragmentation, and avoiding unnecessary data copying.

In a typical circular buffer, two indices—a head and a tail—control data flow. The head marks where a producer (or writer) inserts new data elements, while the tail marks where a consumer (or reader) retrieves data elements. The memory buffers 110, 114, and 120 may be circular buffers that support multiple readers and includes two writer heads. In other words, each circular buffer may have two heads for receiving data. Thus, the circular buffer may support multi-reader synchronization and may be implemented with two distinct writer indices:
alloc_head: tracks image data frames currently being processed; and
write_head: tracks image data frames that have completed processing and are ready for consumption (for output).

At any given time, data elements in the circular buffer exist in one of two states, including processing state (allocated but not yet ready) or a processed state (available for readers). The alloc_head index marks a range of frames currently in processing, while the write_head index tracks a range of frames that have been fully processed and are ready for output. Multiple reader indices (read_heads) track progress for each consumer independently. A buffer's capacity is managed such that the buffer always starts from the reader that is the furthest behind and extends up to the alloc_head index.

Without the optimizations described herein, compression typically takes up 20.96% of the CPU cycles, and the processing pipeline takes up 70.41% of the CPU cycles. For example, the optimizations include vectorizing the bit converter, improving pipeline efficiency, eliminating unnecessary threads, and reducing memory copies. In this unoptimized scenario, the pipeline step accounts for 70.41% of total CPU execution time, with compression contributing to 20.96% of the total CPU execution time. The bit converter alone consumes 38.14% of the total CPU execution time.

With the optimizations described herein, compression takes up 75% of the CPU cycles, and the processing pipeline takes up 9.81% of the CPU cycles. For example, after vectorization, the bit converter's execution time was reduced to 2.85% of the total CPU execution time, representing an improvement of approximately 13x. Consequently, an overall pipeline time reduction from 70.41% to 9.81% was realized in the processing pipeline, achieving a 7× reduction in total CPU execution time. With these optimizations in place, compression becomes the most CPU-intensive step in the software pipeline.

As indicated above, FIG. 1 is provided merely as an example. Other examples may differ from what is described with regard to FIG. 1.

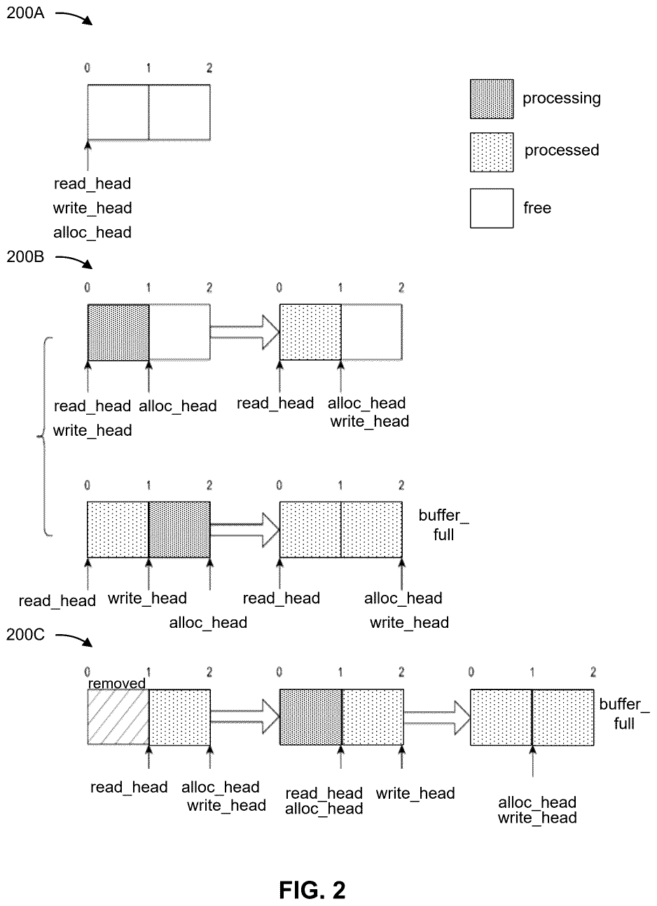

FIG. 2 shows diagrams 200A, 200B, and 200C of an operation flow of a circular buffer with two slots. Initially, as shown in diagram 200A, the circular buffer is empty, and all indices (e.g., read_head index, write_head index, and alloc_head index) are aligned at a same position. To add a new element, the alloc_head index is first incremented, marking a first slot as in processing. Once processing of the first slot is complete, the write_head index advances, indicating that the element is ready for consumption. This process repeats until the circular buffer reaches full capacity, as shown in diagram 200B.

When the circular buffer is full, the circular buffer can either wait for the slowest reader to advance, making room for a new element, or force allocate a new element, overwriting unread data from lagging readers. In diagram 200C, a reader consumes one element while new data is written, demonstrating how the circular buffer wraps around and reuses available slots, maintaining a continuous data flow.

As indicated above, FIG. 2 is provided merely as an example. Other examples may differ from what is described with regard to FIG. 2.

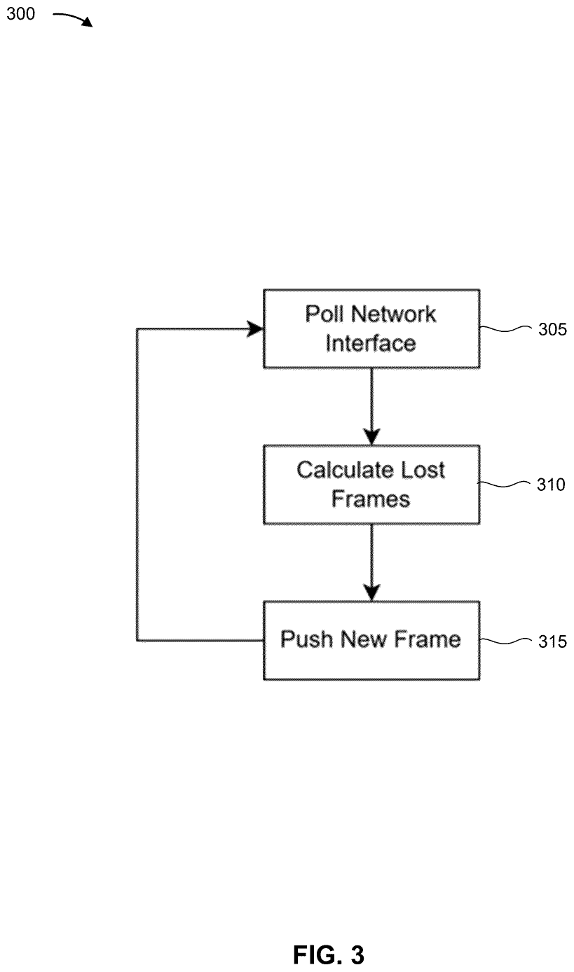

FIG. 3 shows an example 300 of a process flow of the input communication interface 108. The input communication interface 108 manages synchronization between the detector 102 and the input memory buffer 110. When a new acquisition process begins, the input communication interface 108 continuously polls network interfaces connected to the detector's optical links (block 305). Images may be written by the detector 102 directly to the input memory buffer 110 using the RDMA protocol. Upon detecting a new frame, the input communication interface 108 may check for any lost frames by comparing sequence numbers or timestamps (block 310). Finally, the input memory buffer 110 is updated to register an arrival of the new frame (block 315).

As indicated above, FIG. 3 is provided merely as an example. Other examples may differ from what is described with regard to FIG. 3.

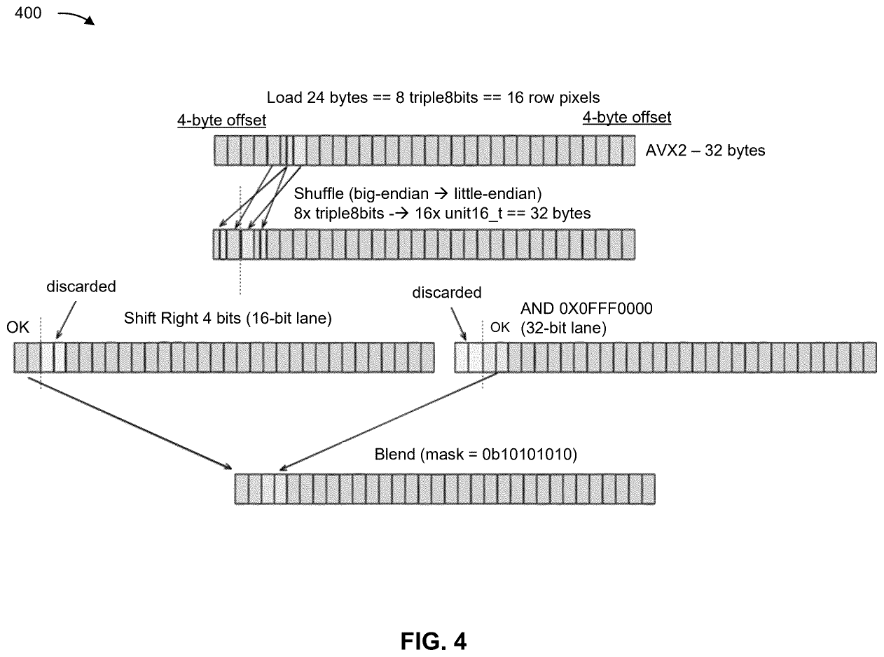

FIG. 4 shows an example 400 of an algorithm for converting 12-bit pixels to 16-bit using advanced vector extensions (AVX), such as AVX2 (256-bit registers). AVX are single instruction, multiple data (SIMD) extensions. The bit converters handle conversion of raw pixels (pixel values) sent by the detector to standard bit depths, such as 16-bit, 32-bit, or any other format required by consumer applications. With increased detector sensor area and frame rate, this area can quickly become a bottleneck of the software pipeline. The bit converters use a set of algorithms that perform the conversion for required formats, leveraging SIMD instructions available in modern hardware.

SIMD is a type of parallel processing. SIMD describes computers that can perform a same operation on multiple data points simultaneously. Modern processors implement a set of SIMD extensions, which are instructions that extend different instructions set architectures (ISAs)—such as x86 and Advanced RISC Machines (ARM), where RISC stands for reduced instruction set computing—and operate on special wide registers.

The algorithms for bit conversion combine vectorized bitwise operations with shuffling and blending to convert the raw pixels to a desired output format (e.g., into standard bit depths).

Compression may ensure that performance and result size of an image to be stored satisfies system requirements. A quantity of bytes for transmission increases as geometry of the detector 102 increases. Additionally, an increase in quantity of bytes reduces a number of frames that can be transmitted per second (e.g., a reduction in frame rate). The number of frames decrease with increase in bytes because transmission links used to transport data is limited by a maximum bandwidth. ZeroMQ is an example of a transmission protocol that can be used to stream image frames from the DCU server.

Compression allows a number of bytes for transmission to be reduced at an expense of processing power and processing time (e.g., CPU execution time) required to compress and decompress the image. Selection of a compression algorithm utilized by the compression engine can be important. For example, the compression algorithm may be selected to properly balance a compression speed and a compression ratio that defines how much smaller a file or a data element will be after compression. The software may enable a user to select between two or more compression algorithms to use for compression to obtain a desired balance between compression speed and compression ratio.

While applying a compression algorithm may be not enough to achieve the highest performance, LZ4, a lossless compression algorithm, is one of the fastest compression algorithms available. However, LZ4, on its own, may not be enough to satisfy speed requirements. In some implementations, division of image data into smaller chunks for parallel compression processing may be implemented. Additionally, a bit shuffle may reduce an entropy of the image data. The division of image data smaller chunks for parallel processing and the bit shuffle, used together with LZ4, may improve the compression of the image data.

As indicated above, FIG. 4 is provided merely as an example. Other examples may differ from what is described with regard to FIG. 4.

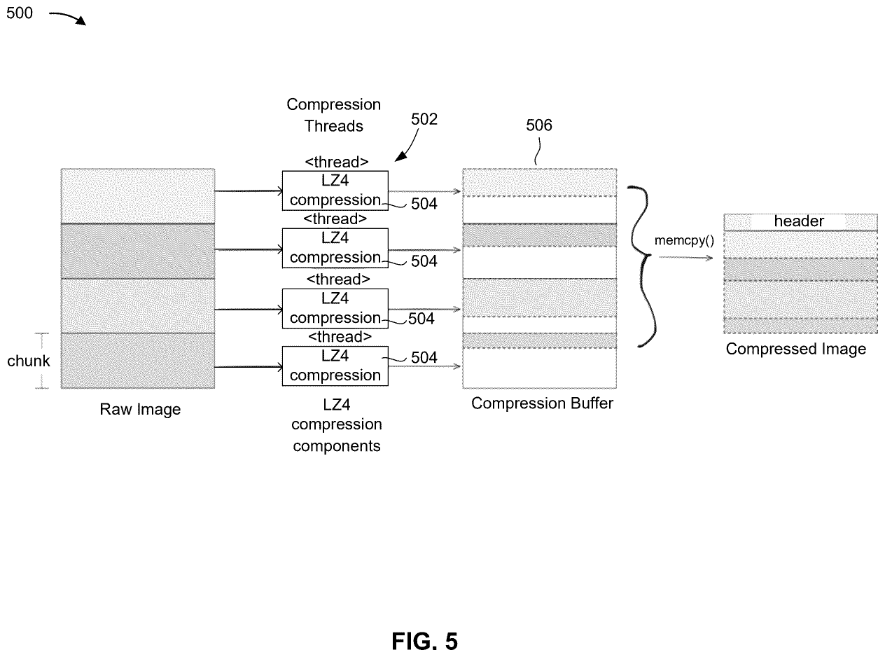

FIG. 5 shows an example 500 of dividing image data into smaller chunks (data chunks) for parallel compression processing. The image data (e.g., an image) may be split into multiple chunks. A plurality of compression threads 502 implemented in the compression engine 118 may be used of performing parallel compression of the multiple chunks. Each compression thread of the plurality of compression threads 502 may include a respective compression component 504, such as an LZ4 compression component, that compresses a respective chunk. Thus, each chunk may be compressed in a separate processing thread to create multiple compressed blocks (e.g., a compressed chunk). Each compressed block may be written into an intermediate compression buffer 506. At best, a compressed block will contain only one LZ4 token, which is less than 8 bytes. When a chunk has high entropy, an output block will be slightly larger than an original chunk due to the fact that LZ4 output tokens carry some extra bytes as metadata (LZ4 metadata). The intermediate compression buffer 506 allows for plurality of compression threads 502 to work in parallel, writing to non-overlapping memory. Finally, compressed blocks may be concatenated into a final compressed image to be streamed (transmitted) by the output communication interface 116. The final compressed image may be first be sent from the intermediate compression buffer 506 to the compressed output memory buffer 120, which then provides the final compressed image to the output communication interface 116.

As indicated above, FIG. 5 is provided merely as an example. Other examples may differ from what is described with regard to FIG. 5.

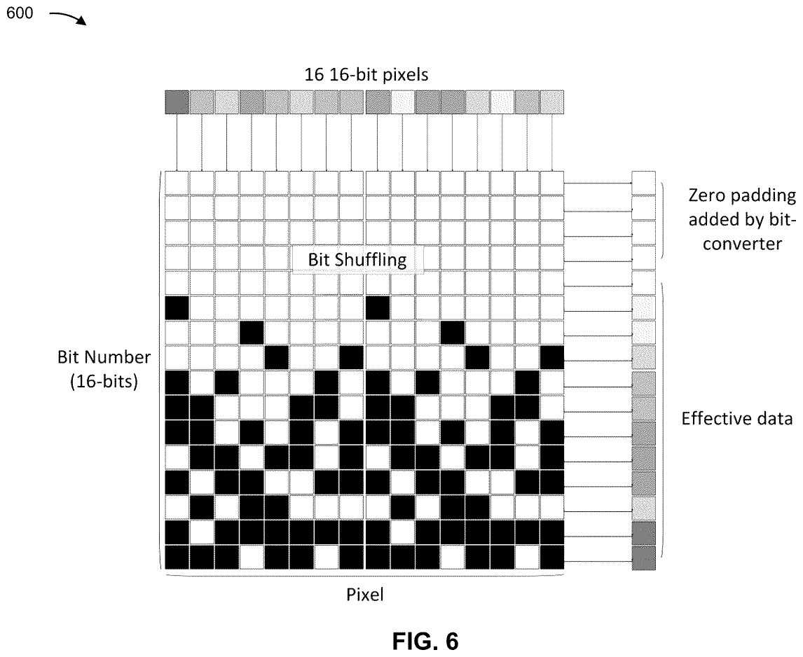

FIG. 6 shows an example 600 of a bit shuffle. The compression engine 118 may perform a bit shuffle to improve the compression described in connection with FIG. 5. Instead of storing data in its original form, bit shuffling reorganizes the bits of the data across multiple values to enhance redundancy and reduce entropy. The bit shuffling transposes a bit matrix making all the least significant bits (LSBs) grouped together, all the second least significant bits grouped together, . . . , and all the most significant bits (MSBs) grouped together, exposing patterns and similarities between the numbers. Bit-shuffling may be performed by the compression engine 118 prior to performing compression.

Finally, to achieve the best performance of the compression, the two techniques described in connection with FIGS. 5 and 6 and the LZ4 algorithm are used in combination. In some implementations, a data compression library Blosc, a blocking, shuffling, and lossless compression library, may be used. The data compression library Blosc (e.g., a high-performance data compressor implemented by a processor) uses a blocking technique to reduce activity in a memory bus as much as possible. In short, this technique works by dividing datasets into blocks that are small enough to fit in caches of modern processors and perform compression/decompression in the caches. The data compression library Blosc also leverages, if available, SIMD instructions (SSE2, AVX2) and multi-threading capabilities of CPUs, to accelerate the compression/decompression process to a maximum. The data compression library Blosc also has an option to implement the bit shuffle algorithm and supports LZ4.

Thus, the data compression library Blosc, LZ4 compression and a bit shuffle may enable the compression engine 118 to achieve high performance.

As indicated above, FIG. 6 is provided merely as an example. Other examples may differ from what is described with regard to FIG. 6.

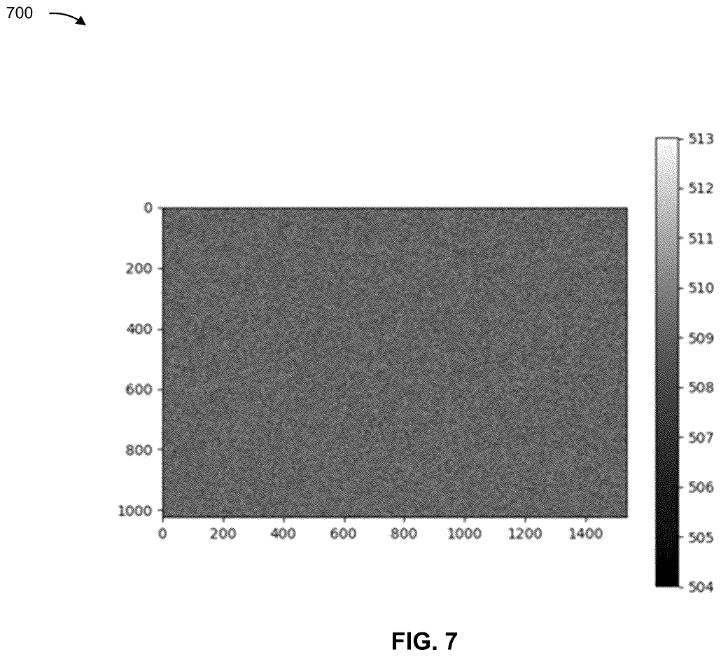

FIG. 7 shows a sample synthetic image 700 used for a compression benchmark.

A compression benchmark uses synthetic images generated using a Poisson probability mass function, ƒ(k; λ), to model the arrival of photons in the detector (Equation 1), where k is the number of photons, λ is the expected number of photons in a given time interval and e is the Euler's number.

$$f ( k ; λ ) = P ( X = k ) = \frac{{λ}^{k} {e}^{- λ}}{k !} \qquad ( Equation 1 )$$

As indicated above, FIG. 7 is provided merely as an example. Other examples may differ from what is described with regard to FIG. 7.

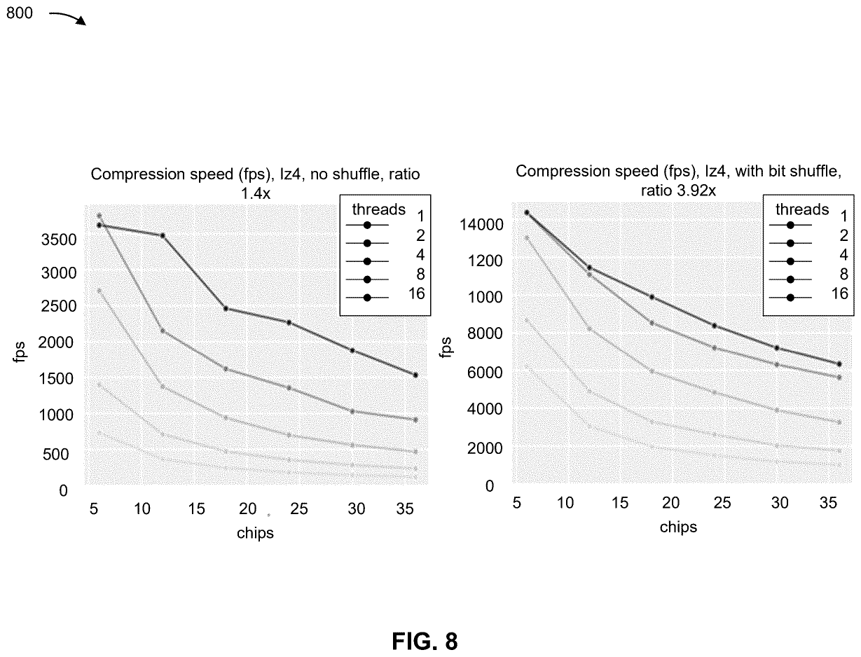

FIG. 8 shows an example 800 of a comparison of a compression speed with varying number of threads and sensor area, with and without applying bit-shuffling technique prior to compressing. In particular, FIG. 8 shows a comparison of compression speed with bit-shuffling off (left) and with bit-shuffling on (right), considering resolutions of 64 kpixels (kilopixels) (6 chips) up to 2.25 Mpixels (megapixels) (36 chips). Applying a bit-shuffle prior to compression achieved increased speeds up to ~4× (e.g., four times faster) for a detector relative to performing the compression without bit-shuffling.

By changing the k parameter in Equation 1, synthetic images were generated with varying pixel intensities, simulating a threshold scan, which is a process that is commonly executed to characterize detector chips.

As indicated above, FIG. 8 is provided merely as an example. Other examples may differ from what is described with regard to FIG. 8.

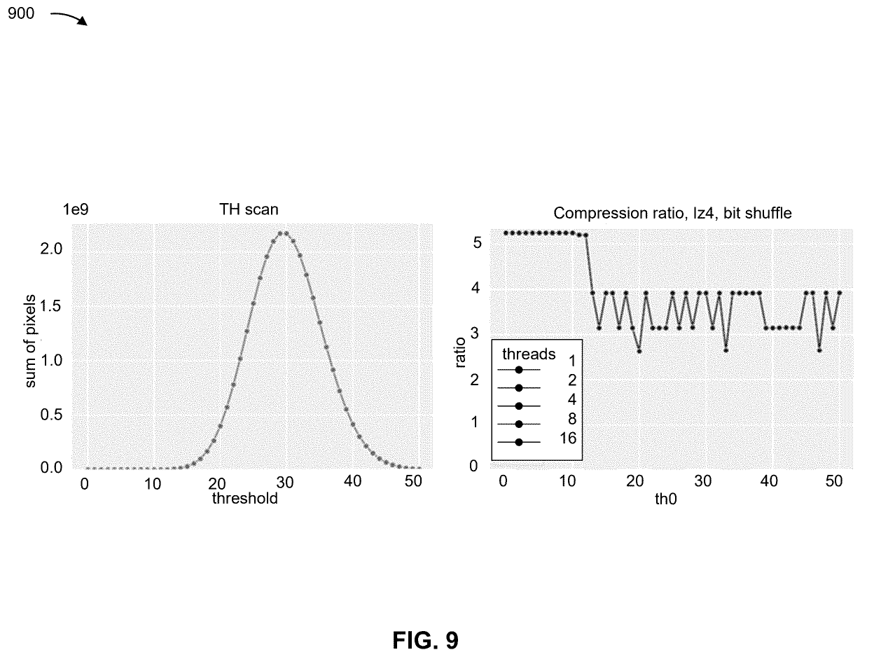

FIG. 9 shows an example 900 of a threshold scan (left) and corresponding compression ratios (right) for a detector frame resolution, applying bit-shuffling before compression. Thus, FIG. 9 shows a threshold scan on the synthetic images with the detector frame resolution to obtain the corresponding compression ratios for different noise levels. The worst compression ratio obtained was 2.63.

As indicated above, FIG. 9 is provided merely as an example. Other examples may differ from what is described with regard to FIG. 9.

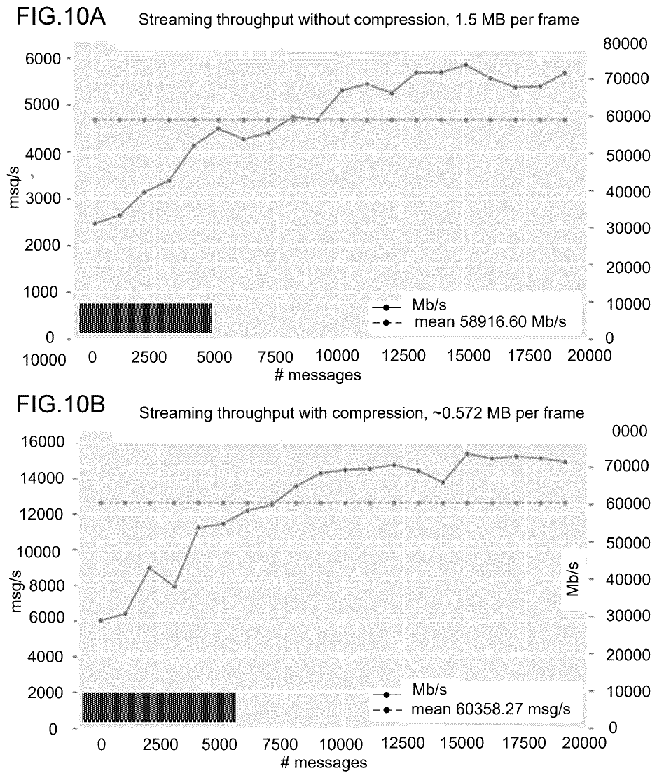

FIGS. 10A and 10B show examples of a streaming benchmark for photon-counting detector frames. In FIG. 10A, the photon-counting detector frames are streamed with compression disabled. In FIG. 10B, the photon-counting detector frames are streamed with compression enabled

The streaming benchmark measures a number of frames per second that can be streamed to a local address as a frame count increases. A plot in FIG. 10A assumes a fixed frame size of 1.5 MB—image of a photon-counting detector—without compression, representing a worst-case streaming scenario, where no data reduction is applied. This evaluation provides a baseline for assessing the system's raw throughput under maximum bandwidth demand. FIG. 10B considers a scenario where compression is applied before streaming the photon-counting detector frames.

Considering a worst case obtained in the results shown in FIG. 9, photon-counting detector frames would be compressed with a 2.63× compression ratio, resulting in a size of approximately 0.572 MB. The DCU server 104, with compression enabled, can stream this amount of data at an average rate of 12615.15 frames per second, as illustrated in FIG. 10B, which is an improvement of about 2.7× with respect to a scenario with compression disabled in FIG. 10A.

As indicated above, FIGS. 10A and 10B are provided merely as examples. Other examples may differ from what is described with regard to FIGS. 10A and 10B.

The following provides an overview of some Aspects of the present disclosure:
Aspect 1: A detector control unit (DCU) server, comprising: an input memory buffer comprising a first plurality of memory locations, the input memory buffer configured to store raw image frames; an input communication interface configured to be communicatively coupled to a photon-counting detector for receiving the raw image frames from the photon-counting detector and provide the raw image frames to assigned memory locations of the first plurality of memory locations of the input memory buffer; a bit-conversion processor coupled to the input memory buffer for receiving the raw image frames from the input memory buffer, wherein the bit-conversion processor includes a plurality of processing threads for performing parallel bit-conversions, and wherein the bit-conversion processor is configured to convert pixel values of each raw image frame of the raw image frames from a raw bit depth to a higher bit depth to generate bit-converted image frames; an output memory buffer configured to receive the bit-converted image frames from the bit-conversion processor, the output memory buffer comprising a second plurality of memory locations for storing the bit-converted image frames; and an output communication interface configured to receive the bit-converted image frames from the output memory buffer, and stream the bit-converted image frames out from the DCU server to an external network.
Aspect 2: The DCU server of Aspect 1, wherein the plurality of processing threads are configured to convert pixel values, from the raw bit depth to the higher bit depth, of a raw image frame of the raw image frames in parallel.
Aspect 3: The DCU server of any of Aspects 1-2, wherein the bit-conversion processor is configured to serially receive the raw image frames from the input memory buffer.
Aspect 4: The DCU server of any of Aspects 1-3, wherein the input communication interface comprises a plurality of parallel optical links for receiving the raw image frames, and wherein the input communication interface is configured to continuously poll the plurality of parallel optical links for detecting a newly-received raw image frame.
Aspect 5: The DCU server of Aspect 4, wherein the input communication interface is configured check for a lost raw image frame by comparing sequence numbers or timestamps of the raw image frames.
Aspect 6: The DCU server of any of Aspects 1-5, wherein the input communication interface is configured to manage synchronization between the photon-counting detector and the input memory buffer.
Aspect 7: The DCU server of Aspect 6, wherein the input communication interface is configured to write the raw image frames into the input memory buffer using a remote direct access memory (RDMA) protocol.
Aspect 8: The DCU server of any of Aspects 1-7, wherein the input memory buffer is a circular buffer comprising a first writer head index, a second writer head index, and one or more reader head indices, wherein the first writer head index indicates a first range of raw image frames currently being processed, wherein the second writer head index follows the first writer head index and indicates a second range of raw image frames that have been processed and are ready to be read out from the input memory buffer, and wherein each reader head index of the one or more reader head indices is configured to advance through the input memory buffer to read out a raw image frame in the second range of raw image frames.
Aspect 9: The DCU server of any of Aspects 1-8, wherein the output memory buffer is a circular buffer comprising a first writer head index, a second writer head index, and one or more reader head indices, wherein the first writer head index indicates a first range of bit-converted image frames currently being processed, wherein the second writer head index follows the first writer head index and indicates a second range of bit-converted image frames that have been processed and are ready to be read out from the output memory buffer, and wherein each reader head index of the one or more reader head indices is configured to advance through the output memory buffer to read out a bit-converted image frame in the second range of bit-converted image frames.
Aspect 10: The DCU server of any of Aspects 1-9, wherein the bit-conversion processor is configured to utilize single instruction, multiple data (SIMD) processing, associated with the plurality of processing threads, for converting the raw image frames into the bit-converted image frames.
Aspect 11: The DCU server of Aspect 10, wherein the bit-conversion processor is configured to combine vectorized bitwise operations with shuffling and blending to covert the pixel values from the raw bit depth to the higher bit depth.
Aspect 12: The DCU server of any of Aspects 1-11, further comprising: a compression engine configured to receive the bit-converted image frames from the output memory buffer, and compress the bit-converted image frames into compressed image frames; and a compression output memory buffer configured to receive the compressed image frames from the compression engine, the compressed output memory buffer comprising a third plurality of memory locations for storing the compressed image frames, wherein the output communication interface is configured to receive the compressed image frames from the compression output memory buffer, and selectively stream the bit-converted image frames or the compressed image frames out from the DCU server to the external network.
Aspect 13: The DCU server of Aspect 12, wherein the compression engine is configured to utilize a lossless data compression algorithm in combination with bit shuffling to compress the bit-converted image frames into the compressed image frames.
Aspect 14: The DCU server of Aspect 13, wherein the lossless data compression algorithm is a Lempel-Ziv-4 (LZ4) data compression algorithm.
Aspect 15: The DCU server of Aspect 12, wherein the compression engine is configured to, for each bit-converted image frame of the bit-converted image frames: divide a bit-converted image frame into chunks for parallel processing of the chunks, compress the chunks, in separate compression threads of a plurality of compression threads, into compressed blocks, write each compressed block of the compressed blocks into an intermediate compression buffer, and concatenate the compressed blocks into a compressed image frame.
Aspect 16: The DCU server of Aspect 12, wherein the output communication interface is configured to selectively stream the bit-converted image frames or the compressed image frames out from the DCU server to the external network based on an asynchronous messaging mechanism.
Aspect 17: A detector control unit (DCU) server, comprising: an input memory buffer comprising a first plurality of memory locations, the input memory buffer configured to store raw image frames; an input communication interface configured to be communicatively coupled to a photon-counting detector for receiving the raw image frames from the photon-counting detector and provide the raw image frames to assigned memory locations of the first plurality of memory locations of the input memory buffer; a bit-conversion processor coupled to the input memory buffer for receiving the raw image frames from the input memory buffer, wherein the one or more processors include a plurality of processing threads for performing parallel bit-conversions, and wherein the one or more processors are configured to convert pixel values of each raw image frame of the raw image frames from a raw bit depth to a higher bit depth to generate bit-converted image frames; an output memory buffer configured to receive the bit-converted image frames from the bit-conversion processor, the output memory buffer comprising a second plurality of memory locations for storing the bit-converted image frames; a compression engine configured to receive the receive the bit-converted image frames from the output memory buffer, and compress the bit-converted image frames into compressed image frames; a compression output memory buffer configured to receive the compressed image frames from the compression engine, the compressed output memory buffer comprising a third plurality of memory locations for storing the compressed image frames; and an output communication interface configured to receive the compressed image frames from the compression output memory buffer, and stream the compressed image frames out from the DCU server to an external network.
Aspect 18: The DCU server of Aspect 17, wherein the plurality of processing threads are configured to convert pixel values, from the raw bit depth to the higher bit depth, of a raw image frame of the raw image frames in parallel, and wherein the compression engine comprises a plurality of compression threads configured to compress chunks of a bit-converted image frame, of the bit-converted image frames, in parallel.
Aspect 19: The DCU server of Aspect 18, wherein the bit-conversion processor is configured to utilize single instruction, multiple data (SIMD) processing, associated with the plurality of processing threads, for converting the raw image frames into the bit-converted image frames.
Aspect 20: A method of processing raw image frames from a photon-counting detector, the method comprising: receiving, by an input communication interface, the raw image frames from the photon-counting detector; providing, by the input communication interface, the raw image frames to assigned memory locations of a first plurality of memory locations of an input memory buffer; reading out, by a bit-conversion processor, the raw image frames from the input memory buffer to the bit-conversion processor; converting, by the bit-conversion processor, the raw image frames into bit-converted image frames, wherein converting the raw image frames into bit-converted image frames includes: processing each raw image frame of the raw image frames in a plurality of processing threads, of the bit-conversion processor, for performing parallel bit-conversions such that pixel values of each raw image frame of the raw image frames are converted from a raw bit depth to a higher bit depth to generate a bit-converted image frame; and streaming, by an output communication interface, the bit-converted image frames or compressed-versions of the bit-converted image frames to an external network.
Aspect 21: A system configured to perform one or more operations recited in one or more of Aspects 1-20.
Aspect 22: An apparatus comprising means for performing one or more operations recited in one or more of Aspects 1-20.
Aspect 23: A non-transitory computer-readable medium storing a set of instructions, the set of instructions comprising one or more instructions that, when executed by a device, cause the device to perform one or more operations recited in one or more of Aspects 1-20.
Aspect 24: A computer program product comprising instructions or code for executing one or more operations recited in one or more of Aspects 1-20.

The foregoing disclosure provides illustration and description, but is not intended to be exhaustive or to limit the implementations to the precise forms disclosed. Modifications and variations may be made in light of the above disclosure or may be acquired from practice of the implementations. Furthermore, any of the implementations described herein may be combined unless the foregoing disclosure expressly provides a reason that one or more implementations may not be combined.

As used herein, the term “component” is intended to be broadly construed as hardware, firmware, and/or a combination of hardware and software. It will be apparent that systems and/or methods described herein may be implemented in different forms of hardware, firmware, or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be designed to implement the systems and/or methods based on the description herein.

Even though particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set. As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiple of the same item.

When a component or one or more components (e.g., a laser emitter or one or more laser emitters) is described or claimed (within a single claim or across multiple claims) as performing multiple operations or being configured to perform multiple operations, this language is intended to broadly cover a variety of architectures and environments. For example, unless explicitly claimed otherwise (e.g., via the use of “first component” and “second component” or other language that differentiates components in the claims), this language is intended to cover a single component performing or being configured to perform all of the operations, a group of components collectively performing or being configured to perform all of the operations, a first component performing or being configured to perform a first operation and a second component performing or being configured to perform a second operation, or any combination of components performing or being configured to perform the operations. For example, when a claim has the form “one or more components configured to: perform X; perform Y; and perform Z,” that claim should be interpreted to mean “one or more components configured to perform X; one or more (possibly different) components configured to perform Y; and one or more (also possibly different) components configured to perform Z.”

No element, act, or instruction used herein should be construed as critical or essential unless explicitly described as such. Also, as used herein, the articles “a” and “an” are intended to include one or more items, and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, or a combination of related and unrelated items), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A detector control unit (DCU) server, comprising:
an input memory buffer comprising a first plurality of memory locations, the input memory buffer configured to store raw image frames;
an input communication interface configured to be communicatively coupled to a photon-counting detector for receiving the raw image frames from the photon-counting detector and provide the raw image frames to assigned memory locations of the first plurality of memory locations of the input memory buffer;
a bit-conversion processor coupled to the input memory buffer for receiving the raw image frames from the input memory buffer, wherein the bit-conversion processor includes a plurality of processing threads for performing parallel bit-conversions, and wherein the bit-conversion processor is configured to convert pixel values of each raw image frame of the raw image frames from a raw bit depth to a higher bit depth to generate bit-converted image frames;
an output memory buffer configured to receive the bit-converted image frames from the bit-conversion processor, the output memory buffer comprising a second plurality of memory locations for storing the bit-converted image frames; and
an output communication interface configured to receive the bit-converted image frames from the output memory buffer, and stream the bit-converted image frames out from the DCU server to an external network.

2. The DCU server of claim 1, wherein the plurality of processing threads are configured to convert pixel values, from the raw bit depth to the higher bit depth, of a raw image frame of the raw image frames in parallel.

3. The DCU server of claim 1, wherein the bit-conversion processor is configured to serially receive the raw image frames from the input memory buffer.

4. The DCU server of claim 1, wherein the input communication interface comprises a plurality of parallel optical links for receiving the raw image frames, and wherein the input communication interface is configured to continuously poll the plurality of parallel optical links for detecting a newly-received raw image frame.

5. The DCU server of claim 4, wherein the input communication interface is configured check for a lost raw image frame by comparing sequence numbers or timestamps of the raw image frames.

6. The DCU server of claim 1, wherein the input communication interface is configured to manage synchronization between the photon-counting detector and the input memory buffer.

7. The DCU server of claim 6, wherein the input communication interface is configured to write the raw image frames into the input memory buffer using a remote direct access memory (RDMA) protocol.

8. The DCU server of claim 1, wherein the input memory buffer is a circular buffer comprising a first writer head index, a second writer head index, and one or more reader head indices,
wherein the first writer head index indicates a first range of raw image frames currently being processed,
wherein the second writer head index follows the first writer head index and indicates a second range of raw image frames that have been processed and are ready to be read out from the input memory buffer, and
wherein each reader head index of the one or more reader head indices is configured to advance through the input memory buffer to read out a raw image frame in the second range of raw image frames.

9. The DCU server of claim 1, wherein the output memory buffer is a circular buffer comprising a first writer head index, a second writer head index, and one or more reader head indices,
wherein the first writer head index indicates a first range of bit-converted image frames currently being processed,
wherein the second writer head index follows the first writer head index and indicates a second range of bit-converted image frames that have been processed and are ready to be read out from the output memory buffer, and
wherein each reader head index of the one or more reader head indices is configured to advance through the output memory buffer to read out a bit-converted image frame in the second range of bit-converted image frames.

10. The DCU server of claim 1, wherein the bit-conversion processor is configured to utilize single instruction, multiple data (SIMD) processing, associated with the plurality of processing threads, for converting the raw image frames into the bit-converted image frames.

11. The DCU server of claim 10, wherein the bit-conversion processor is configured to combine vectorized bitwise operations with shuffling and blending to covert the pixel values from the raw bit depth to the higher bit depth.

12. The DCU server of claim 1, further comprising:
a compression engine configured to receive the bit-converted image frames from the output memory buffer, and compress the bit-converted image frames into compressed image frames; and
a compression output memory buffer configured to receive the compressed image frames from the compression engine, the compressed output memory buffer comprising a third plurality of memory locations for storing the compressed image frames,
wherein the output communication interface is configured to receive the compressed image frames from the compression output memory buffer, and selectively stream the bit-converted image frames or the compressed image frames out from the DCU server to the external network.

13. The DCU server of claim 12, wherein the compression engine is configured to utilize a lossless data compression algorithm in combination with bit shuffling to compress the bit-converted image frames into the compressed image frames.

14. The DCU server of claim 13, wherein the lossless data compression algorithm is a Lempel-Ziv-4 (LZ4) data compression algorithm.

15. The DCU server of claim 12, wherein the compression engine is configured to, for each bit-converted image frame of the bit-converted image frames:
divide a bit-converted image frame into chunks for parallel processing of the chunks, compress the chunks, in separate compression threads of a plurality of compression threads, into compressed blocks,
write each compressed block of the compressed blocks into an intermediate compression buffer, and
concatenate the compressed blocks into a compressed image frame.

16. The DCU server of claim 12, wherein the output communication interface is configured to selectively stream the bit-converted image frames or the compressed image frames out from the DCU server to the external network based on an asynchronous messaging mechanism.

17. A detector control unit (DCU) server, comprising:
an input memory buffer comprising a first plurality of memory locations, the input memory buffer configured to store raw image frames;
an input communication interface configured to be communicatively coupled to a photon-counting detector for receiving the raw image frames from the photon-counting detector and provide the raw image frames to assigned memory locations of the first plurality of memory locations of the input memory buffer;
a bit-conversion processor coupled to the input memory buffer for receiving the raw image frames from the input memory buffer, wherein the one or more processors include a plurality of processing threads for performing parallel bit-conversions, and wherein the one or more processors are configured to convert pixel values of each raw image frame of the raw image frames from a raw bit depth to a higher bit depth to generate bit-converted image frames;
an output memory buffer configured to receive the bit-converted image frames from the bit-conversion processor, the output memory buffer comprising a second plurality of memory locations for storing the bit-converted image frames;
a compression engine configured to receive the receive the bit-converted image frames from the output memory buffer, and compress the bit-converted image frames into compressed image frames;
a compression output memory buffer configured to receive the compressed image frames from the compression engine, the compressed output memory buffer comprising a third plurality of memory locations for storing the compressed image frames; and
an output communication interface configured to receive the compressed image frames from the compression output memory buffer, and stream the compressed image frames out from the DCU server to an external network.

18. The DCU server of claim 17, wherein the plurality of processing threads are configured to convert pixel values, from the raw bit depth to the higher bit depth, of a raw image frame of the raw image frames in parallel, and
wherein the compression engine comprises a plurality of compression threads configured to compress chunks of a bit-converted image frame, of the bit-converted image frames, in parallel.

19. The DCU server of claim 18, wherein the bit-conversion processor is configured to utilize single instruction, multiple data (SIMD) processing, associated with the plurality of processing threads, for converting the raw image frames into the bit-converted image frames.

20. A method of processing raw image frames from a photon-counting detector, the method comprising:
receiving, by an input communication interface, the raw image frames from the photon-counting detector;
providing, by the input communication interface, the raw image frames to assigned memory locations of a first plurality of memory locations of an input memory buffer;
reading out, by a bit-conversion processor, the raw image frames from the input memory buffer to the bit-conversion processor;
converting, by the bit-conversion processor, the raw image frames into bit-converted image frames, wherein converting the raw image frames into bit-converted image frames includes:
processing each raw image frame of the raw image frames in a plurality of processing threads, of the bit-conversion processor, for performing parallel bit-conversions such that pixel values of each raw image frame of the raw image frames are converted from a raw bit depth to a higher bit depth to generate a bit-converted image frame; and
streaming, by an output communication interface, the bit-converted image frames or compressed-versions of the bit-converted image frames to an external network.
